SEQUENCE LISTING (1 sequence)

SEQ ID NO: 1 - protein, 360 aa, Homo sapiens
MALSRVCWARSAVWGSAVTPGHFVTRRLQLGRSGLAWGAPRSSKLHLSPKADVKNLMSYVVTKTKAINGKYHRFLGRHFPRFYILYTIFMKGLQMLWADAKKARRIKTNMWKHNIKFHQLPYREMEHLRQFRQDVTKCLFLGIISIPPFANYLVFLLMYLFPRQLLIRHFWTPKQQTDFLDIYHAFRKQSHPEIISYLEKVIPLISDAGLRWRLTDLCTKIQRGTHPAIHDILALRECFSNHPLGMNQLQALHVKALSRAMLLTSYLPPPLLRHRLKTHTTVIHQLDKALAKLGIGQLTAQEVKSACYLRGLNSTHIGEDRCRTWLGEWLQISCSLKEAELSLLLHNVVLLSTNYLGTRR

(19) United States
(12) Patent Application Publication
(10) Pub. No.: US 2010/0205681 A1
(43) Pub. Date: Aug. 12, 2010

(54) PROGNOSTIC MARKER FOR BREAST CANCER AND COMPOSITION FOR INDUCING OBESITY COMPRISING HCCR-1

(76) Inventors: Hyun-Kee Kim, Seoul (KR); Jin-woo Kim, Seoul (JP)

Correspondence Address: ROBERT E. BUSHNELL & LAW FIRM, 2029 K STREET NW, SUITE 600, WASHINGTON, DC, 20006-1004, US

(21) Appl. No.: 12/309,102

(22) PCT Filed: Jul. 6, 2007
(86) PCT No.: PCT/KR2007/003293

§ 371 Date: Feb. 2, 2009

(30) Foreign Application Priority Data
Jul. 7, 2006 (KR) 10-2006-0064035

(51) Int. Cl. A01K 67/027; C07K 14/435; G01N 33/53

(52) U.S. Cl. 800/15; 530/350; 436/86; 800/14; 800/18; 800/16; 800/17

(57) ABSTRACT

A prognostic marker for breast cancer and a composition for inducing obesity are provided, wherein said marker and said composition comprise HCCR-1.

### BACKGROUND OF THE INVENTION

1. Field of the Invention

The present invention relates to a prognostic marker for breast cancer and/or a composition for inducing obesity, wherein said marker and said composition comprise HCCR-1.

2. Description of the Related Art

Prognosis of breast cancer is determined mainly by the presence of axillary lymph node metastasis. However, after 10 years since local or topical treatment, breast cancer relapses in about a third of breast cancer women with negative lymph node and it does not occur in a third of patients with positive lymph node. Recently, the percentage of breast cancer diagnosed at an early stage is growing. Usually subjecting these patients to systemic therapy often becomes over-treatment. According to St. Gallen and NIH consensus, about 70 to 80% of patients at Stages I and II do not show distant metastasis without auxiliary therapy, and may be suffering from side effects. This fact indicates that there is a need for a more sensitive and specific prognostic analysis which can significantly reduce the number of patients undergoing unnecessary treatment. In some studies, it has been found that tumor size, or lymph or vascular invasion has a significant prognostic value. The quantitative pathological features such as nuclear shape, DNA content and proliferative activity allow for distinction of tumors which have a high potential for micrometastasis. Even though the genetic changes of known molecules which affect the outcome of patients include Her2/NEU overexpression, DNA amplification, p53 mutation, ER/PR status and the like, since metastasis cascades involve a number of complex steps, it is still insufficient to assess prognosis only with such factors.

Also, when developing a product such as a dietary food or beverage or a treatment for hyperlipidemia, obesity-induced experimental mice and the like can be useful for use as obesity-induced mice and the like which are necessary to evaluate the efficacies of such product.

### SUMMARY OF THE INVENTION

The present invention is made from the above-described point of view.

Thus, the object of the present invention is to provide a prognostic marker for breast cancer.

Another object of the present invention is to provide a composition for inducing obesity.

In order to achieve the above object, the present invention provides a composition for use as a breast cancer prognostic marker wherein said composition comprises HCCR-1 protein.

For the present invention, it is preferable for said composition to further comprise one or more agents selected from the group consisting of, but not limited to, ER, PR, p53 genotype and HER2 protein.

Also, for the present invention, said HCCR-1 protein may possibly be any protein which is substantially equivalent to the protein comprising the amino acid sequence set forth in SEQ ID NO: 1. In this context, “substantially equivalent” means any protein which has been mutated by substitution, deletion, addition, etc. of a certain moiety of the protein sequence set forth in SEQ ID NO:1 but retains the properties of HCCR-1, as well as its fragments.

The present invention also provides a composition for inducing obesity wherein said composition comprises HCCR-1 protein.

The present invention also provides a transgenic, obese non-human mammal which has been transformed with HCCR-1 protein.

In the present invention, said animal is preferably, but not limited to, a transgenic, obese non-human mammalian animal selected from the group consisting of mouse, rat, rabbit, sheep, bovine, goat and porcine.

Said HCCR-1 protein may possibly be any protein which is substantially equivalent to the protein comprising the amino acid sequence set forth in SEQ ID NO: 1. In this context, “substantially equivalent” intends to encompass any protein which has been mutated by substitution, deletion, addition, etc. of a certain moiety of the protein sequence set forth in SEQ ID NO:1 but retains the properties of HCCR-1, as well as its fragments.

The present invention will be hereinafter described in detail.

### BRIEF DESCRIPTION OF THE DRAWINGS

FIG. 1 shows HCCR-1 expression and the association between HCCR-1 and ApoE. (a) Immunohistochemical staining of human breast cancers for the expression of ER, PR, HER2, p53 and HCCR-1. All of the breast cancer tissues were derived from pathologically proven invasive ductal carcinomas. Carcinoma cells show positive immunostaining for ER, PR and p53 in the nuclei and for HCCR-1 in the protoplasms (top panel). HER2 immunohistochemical staining shows complete, strong membrane staining (3+) of carcinoma cells (top panel). Negative immunostaining for ER, PR, p53, HER2 and HCCR-1 in breast carcinoma (lower panel). Magnification of originals, ×100. (b) Protein interaction between HCCR-1 and ApoE. HCCR-1 binds ApoE (left panel), and ApoE binds HCCR-1 (right panel). Co-immunoprecipitation was performed from transfected MCF-7 cells which produce HCCR-1 and ApoE proteins. The immunoprecipitation was performed with anti-V5 mAb or anti-Myc mAb. The proteins in the pellets were detected with anti-Myc and anti-V5 mAb, respectively. (c) Localization and co-existence of HCCR-1 and ApoE in MCF-7 cells. C represents protoplasm, N represents nucleus, and M represents mitochondria. Subcellular localization of HCCR-1 (top panel) and its binding protein, ApoE (lower panel). cDNA constructs were designed such that V5 (top panel) and c-Myc (lower panel) were tagged to HCCR-1 and ApoE, respectively. Voltage-dependent anion channel 1 (VDAC1) was used as a mitochondrial marker. (d) Fluorescent microscope. Cells were transiently transfected with pEGFP-HCCR-1 and pEGFP-ApoE using LipofectAMINE 2000 (Invitrogen, Carlsbad, Calif.). Then, the cells were incubated with 25 nM MitoTracker Orange (Molecular Probes, Eugene, Oreg.). For GFP staining, the cells were fixed using ProLong Gold Antifade Reagents (Molecular Probes, Eugene, Oreg.). Fluorescent images were analysed using a Bio-Rad MRC-1024 MP laser scanning confocal microscope (Bio-Rad, Hercules, Calif.).

FIG. 2 shows that HCCR-1 and ApoE are reciprocally regulated in breast cancer tissues. Expression pattern of HCCR-1 (a) and ApoE (b) in human breast tissues and cell lines. Comparison of expression of HCCR-1 mRNA in breast cancer cell lines (BT-474, MCF-7 and MDA-MB-231), fresh primary breast cancer tissues and their corresponding normal counterparts. ‘N’ represents normal breast tissue and ‘C’ represents primary breast cancer tissue. Human beta actin cDNA was used as a control probe (lower panel). (c,d) Expression pattern of ApoE in human tissues and cell lines. Human cancer cell line multiple Northern blot (c) or normal 12-lane multiple-tissue Northern blot (d) was probed with a radiolabeled ApoE cDNA (top panel) or with a human beta actin cDNA control probe (lower panel). (e, f) These pictures show that ApoE plays a role of tumor suppressor in breast cancer. (e) This shows growth inhibitory effect of ApoE in MCF-7 breast cancer cells. Survival of MCF-7 cells was shown after transfection with vector only, HCCR-1 and ApoE, and co-transfection. Data represents the number of viable cells during 10 days of incubation and represents mean±S.D. of triplicate experiments. (f) ApoE cDNA transfection induces apotosis in breast cancer cells. DNAs from the cells which were transfected with vector only, HCCR-1 and ApoE, and co-transfected with the latter two genes, were subjected to electrophoretic analysis. The cells transfected with vector or gene (s) were incubated for 1, 3, 5 and 7 days. DNAs were analysed in a 2% agarose gel and stained with ethidium bromide. (g,h) Secretion of ApoE by MCF-7 cells. ApoE from MCF-7 cells was measured by ELISA. Results represent mean±S.D. of triplicate experiments. ApoE concentrations were determined by sandwich ELISA using a commercial kit (MBL ApoE4/Pan-ApoE ELISA kit, MBL Co., Woburn, Mass.). Human Pan-ApoE (g) or ApoE4 (h) was determined by sandwich ELISA using the MBL ApoE4/Pan-ApoE ELISA kit (MBL Co., Woburn, Mass.).

FIG. 3 shows phenotypic analysis and expression pattern of HCCR-1 and ApoE in HCCR-1 transgenic obese mice. (a) Production of transgenic mice. Transgenic mice were generated using standard pronuclear microinjection. For microinjection, the fragment of transgene, CMV-HCCR-1-bGH was microinjected into the pronuclei of one cell-stage fertilized embryos derived from C57BL/6N (Charles River Japan). Transgenic (T/G) male and non-T/G control male mice (left panel). T/G female and non-T/G control female mice (right panel). (b) Hematoxylin-eosin(HE) staining of peritoneum, liver, pancreas and heart derived from obese and control mice. Male T/G obese mice (top panel), female T/G obese mice (middle panel) and control non-T/G mice (lower panel). Magnification of originals, ×200. (c) Expression level of ApoE and HCCR-1 in various organs of obese and control mice. ApoE (A1.4) mouse monoclonal antibody raised against amino acids 126-191 of ApoE of human organ was used (Santa Cruz biotechnology). (d) Serum profile of ApoE, leptin, cholesterol, triglyceride, insulin and glucose in obese and control mice. Results represent mean±S.D. of triplicate experiments.

### DETAILED DESCRIPTION OF THE INVENTION

The value of HCCR-1 as a prognostic factor for breast cancer has been further proved by its positive correlation with the expression levels of the other known prognostic markers such as steroid receptors (ER and PR), p53 mutant and higher HER2 activity in 30 primary invasive ductal carcinomas of breast.

Also, secretion of ApoE is inhibited by the expression of HCCR-1. In line with this, synthetic siRNA targeting HCCR-1 abrupts the inhibitory effects of HCCR-1 on the secretion of ApoE. Thus, HCCR-1 which interacts with ApoE, the main regulator of lipid metabolism, induced obesity in transgenic mice lines. Obese mice weighed about 3 times more than normal mice. The obese mice showed severe hypercholesterolemia, hypertriglyceridemia and hypoinsulinaemia, although they showed no increase in ApoE or leptin. Also, obese mice had pathological problems in peritoneum, liver, pancreas and heart.

Accordingly, expression of HCCR-1 can be used as a new prognostic marker in combination with already known prognostic factors. HCCR-1 negatively regulates the function of ApoE via physical interaction and ApoE-interacting HCCR-1 induces obesity by inhibiting cholesterol-lowering activity of ApoE.

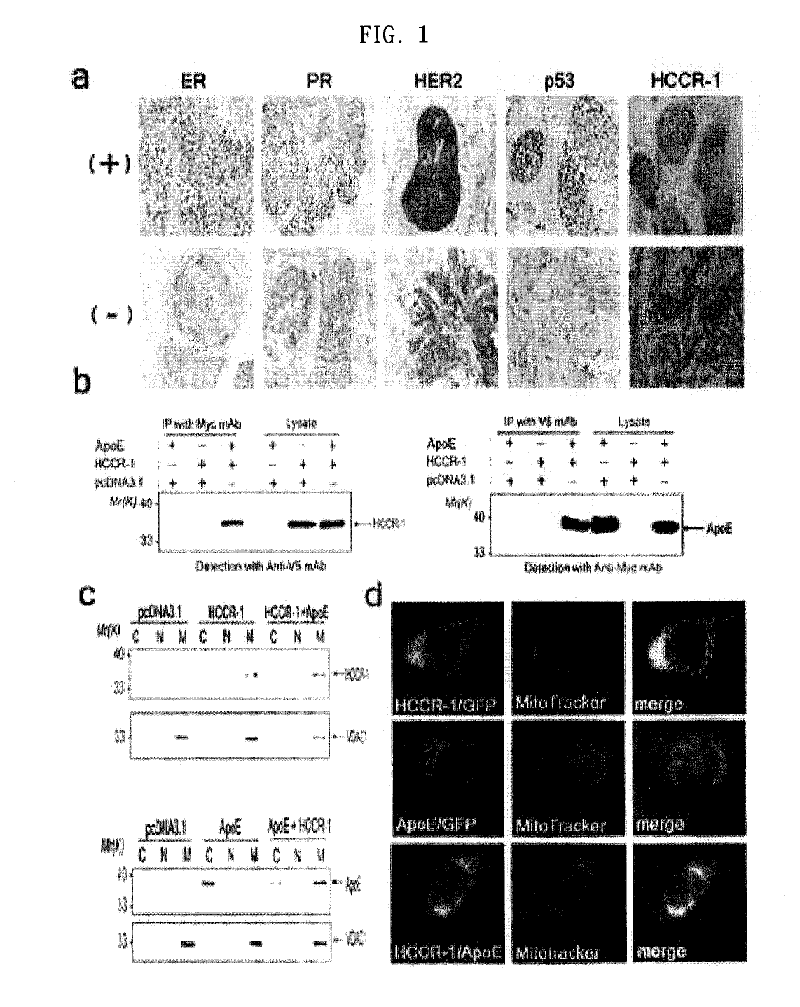

In order to see if the expression of HCCR-1 protein in primary breast cancer tissues is associated with other biomarkers such as ER, PR, p53 genotype and HER2 status, the present inventors measured HCCR-1 level for 30 breast cancer tissue panel together with their normal counterparts (Table 1 and FIG. 1a). Increase of expression level of HCCR-1 was observed in the following order: breast cancer tissue having (ER+/PR+/mutantp53/highHER2), (ER+/PR+/wild p53/intermediate HER2) and having (ER+/PR+/wild p53/low HER2) (Table 1 and FIG. 1a; upper panel). HCCR-1 was not detected in the breast cancer tissue having (ER−/PR−/p53−/lowHER2) (Table 1 and FIG. 1a; lower panel). Thus, these results indicate that the expression of HCCR-1 can be used to expect the prognosis of breast cancer.

TABLE 1
Correlation of expression level of HCCR-1 with known breast
cancer prognostic factors including ER/PR expression, p53
status and HER2 activity

| Number of | ER | | | HER2 | HCCR-1 |
|---|---|---|---|---|---|
| cancer tissues | status | PR status | p53 status | activity | expression |
| 8 | + | + | Mutant | high | very |
| | | | | | strong |
| 8 | + | + | wild type | moderate | strong |
| 5 | + | + | wild type | low | weak |
| 9 | − | − | Null | low | none |

In the above Table, all of the breast cancer tissues were derived from pathologically proven invasive ductal carcinomas.

HCCR-1 and ApoE identified by yeast two-hybrid screening were confirmed in vitro by immunoprecipitation (FIG. 1b). In order to confirm the interaction between HCCR-1 and ApoE by co-immunoprecipitation experiment, the present inventors transfected an ApoE-Myc fusion construct into MCF-7 cells expressing HCCR-1 to which V5 tag is fused. In the co-transfected MCF-7 cells, HCCR-1 protein was specifically co-immunoprecipitated with ApoE (FIG. 1b). Also, HCCR-1 and ApoE proteins were expressed in the MCF-7 cell lysates wherein said MCF-7 cells had been transfected with HCCR-1 and ApoE (FIG. 1b). These results demonstrate that ApoE interacts with HCCR-1 protein.

The present inventors detected the mitochondrial localization of HCCR-1 in the MCF-7 cells (FIG. 1c). On the other hand, ApoE is found only in the cytosol fractions (FIG. 1c; lower panel). However, fractional analysis on the MCF-7 cells in which HCCR-1 and ApoE are co-expressed, indicates that these two proteins are found in the mitochondrial fractions and only a minimal amount of ApoE is found in the cytosols (FIG. 1c; lower panel). These results indicate that ApoE proteins migrate from protoplasm to mitochondria in the co-expressed cells; such data further prove their interaction. Also, fluorescent images show that HCCR-1 exists in mitochondria (FIG. 1d; top panel) and ApoE exists in cytosol (FIG. 1d; second panel). In order to further confirm co-existence of HCCR-1 and ApoE in mitochondria, the co-transfected cells were stained with MitoTracker (Red) (FIG. 1d). The stained cells were analysed with a confocal microscope. When the cells were co-transfected with HCCR-1 and ApoE, ApoEmigrated from cytosol to mitochondria (FIG. 1d; lower panel). These results indicate that the two proteins co-exist in mitochondria.

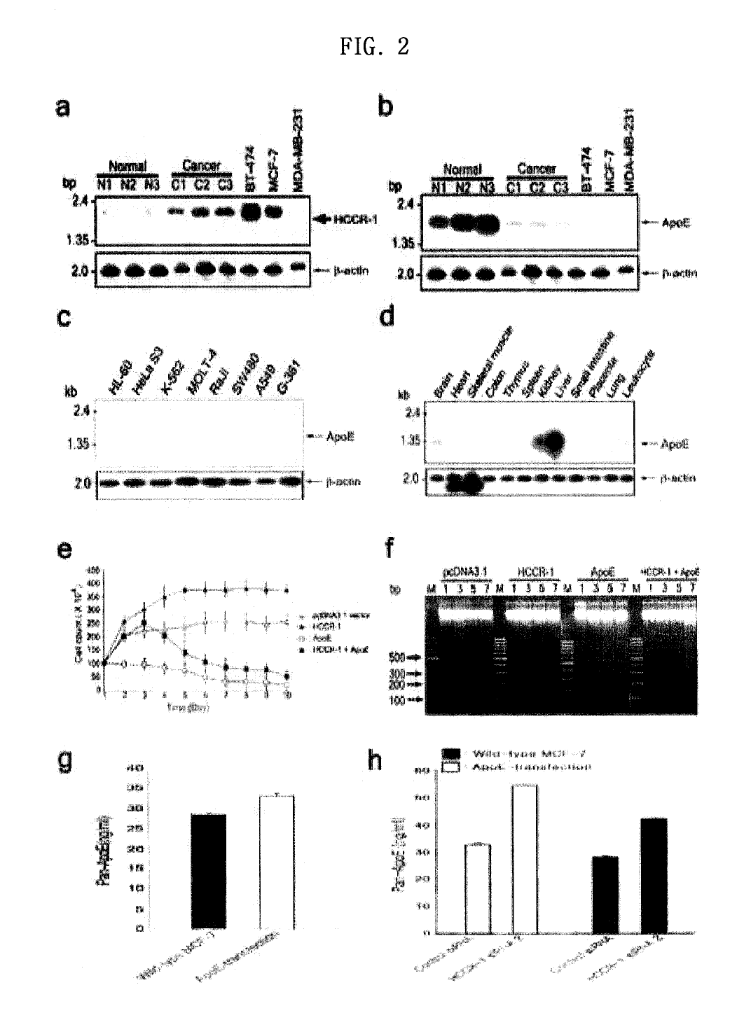

Next, the present inventors investigated expression pattern of HCCR-1 or ApoE in human normal tissues, cancer tissues and cell lines. Northern blot analysis showed that primary breast cancer tissues had an increased expression of HCCR-1 compared to normal tissues (FIG. 2a). HCCR-1 was detected abundantly in BT-474 (ER+/PR+/mutant p53/high HER2) and MCF-7 (ER+/PR+/wild p53/low HER2) cells, whereas it was not detected in MDA-MB-231 (ER−/PR−/p53−/low HER2) cells (FIG. 2a). The expression level of HCCR-1 was higher in MCF-7 cells than in BT-474 cells (FIG. 2a). In contrast to overexpression of HCCR-1 in breast cancer tissues or cells (FIG. 2a), little ApoE expression was observed therein (FIG. 2b). Also, ApoE expression was not detected in other eight cancer cell lines (FIG. 2c). Northern blot analysis showed that ApoE expression was specifically abundant only in liver and kidney among 12 normal tissues tested (FIG. 2d).

In order to determine the growth inhibitory effect of ApoE, ApoE was transfected into breast cancer cell lines. ApoE transfection induced 87% of growth inhibition after 7 days compared to the vector-only transfected control (FIG. 2e). On the other hand, the growth rate of HCCR-1 transfected MCF-7 cells was increased by 45% after 7 days compared to the vector-only transfected control (FIG. 2e). If the HCCR-1-transfected cells were co-transfected with ApoE, the growth was decreased to 66% (FIG. 2e). This inhibitory effect is associated with apoptosis such as DNA fragmentation (FIG. 2f). ApoE serves as a tumor suppressor in breast cancer but this is reversed if HCCR-1 is overexpressed (FIGS. 2e and 2f).

In order to investigate the effect of HCCR-1 on ApoE secretion in the MCF-7 cells, the present inventors made a comparison of Pan-ApoE secretion in the ApoE-transfected MCF-7 cells. ApoE secreted in the cell culture medium was measured by the ApoE4/Pan-ApoE ELISA kit (MBL, Woburn, Mass.). Such kit is based on sandwich ELISA and is capable of measuring Pan-ApoE or ApoE4. After a 24 h incubation with DMEM, the culture medium was collected at both sides to determine the concentration of Pan-ApoE. A small amount (28.4±0.1 ng/ml) of Pan-ApoE was found in the wild-type cells, whereas the amount was increased (33.0±0.6 ng/ml) in the transfected cells (FIG. 2g) (P<0.05). Synthetic siRNA (HCCR-1 siRNA2) which has been designed using the HiPerformance algorithm (Qiagen GmbH, Germany) targeting HCCR-1, induced Pan-ApoE secretion in the MCF-7 breast cancer cells (FIG. 2h). HCCR-1 siRNA-2 which corresponds to nucleotides 579-600 within exon 5 of HCCR-1, induced Pan-ApoE secretion respectively in the wild-type MCF-7 cells and ApoE-transfected MCF-7 cells (FIG. 2h). Pan-ApoE secretion in the ApoE-transfected MCF-7 cells was greatly increased by HCCR1 siRNA-2 (54.5±0.2 ng/ml) compared to the control non-silencing siRNA (33.0±0.3 ng/ml) (FIG. 2h) (P<0.05). In the wild-type MCF-7 cells, HCCR-1 siRNA-2 transfection induced Pan-ApoE secretion in a large amount (42.4±0.3 ng/ml), whereas the secretion was decreased in the control siRNA transfection (28.3±0.3 ng/ml) (FIG. 2h) (P<0.05).

ApoE proteins may regulate the risk of breast cancer via coupling with life habits such as eating or obesity, or with biological factors. Obesity is sharply increasing globally. Although the mechanism, by which obesity causes or promotes cancer, varies with the cancer's site, epidemiological studies show that there is an association between obesity and a range of cancer types. Obesity has been known to increase the rate of breast cancer incidence by 30-50% in women after menopause (Ballard-Barbash, et al. Am Clin Nutr 63(Suppl. 3), 437-441 (1996); Trentham-Dietz, A. et al. Am J Epidemiol 145, 1011-1119 (1997)). Obesity affects the endogenous estrogen metabolism and bioavailability, and thus affects the risk of breast cancer.

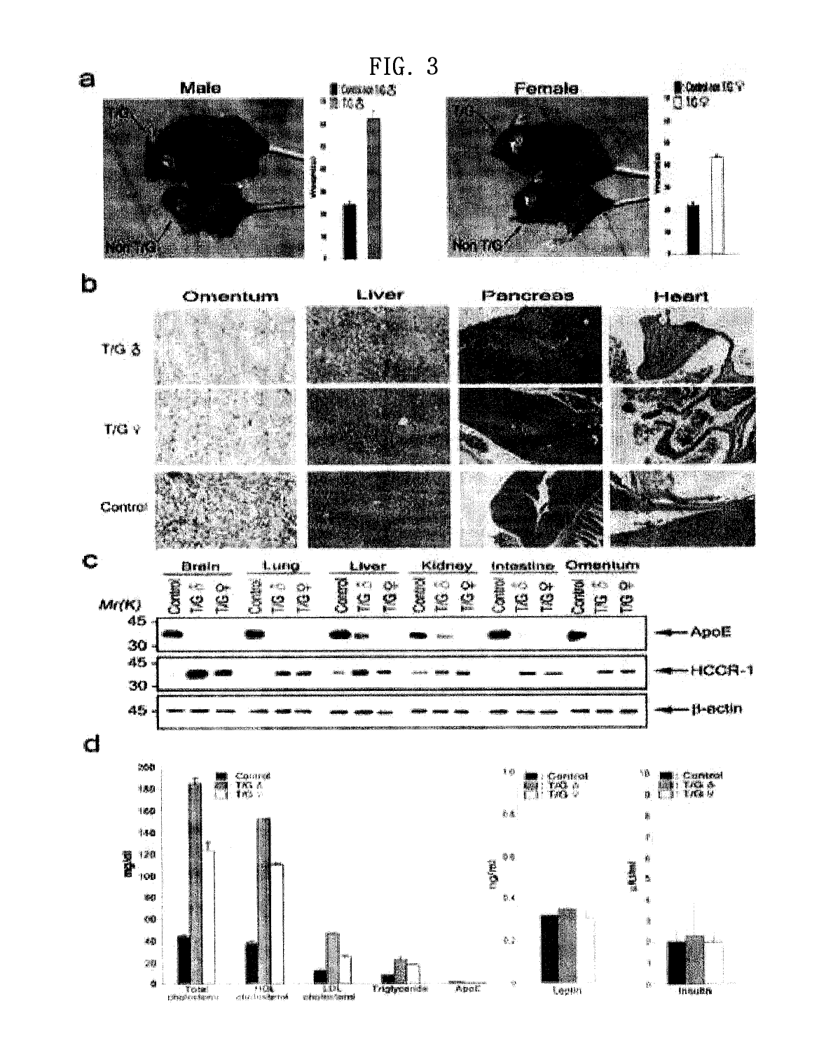

HCCR-1 induced a severe obesity in transgenic mice. Obese mice were bred for more than 3 generations, and their weights were shown to be 3 times heavier than the mice of the same sex and age (FIG. 3a). Particularly, obesity was observed more prominently in male transgenic mice (FIG. 3a; left panel) than in female transgenic mice of the same age (FIG. 3a; right panel). Male obese mice weighed 62.2±3.7 g compared to normal mice of the same age (24.5±10 g) (FIG. 3; left panel) (P<0.0001). Also, there was a significant weight difference between female obese mice (43.1±1.3 g) and normal mice of the same age (21.7±1.5 g) (FIG. 3a; right panel) (P<0.0001).

Obese mice showed pathological conditions including peritoneum, liver, pancreas and heart (FIG. 3b). In transgenic male mice, peritoneum showed a large quantity of fat and adipocyte hypertrophy (FIG. 3b; top panel). Liver showed spreading of microvesicular andmacrovesicular fatty change in hepatocytes (FIG. 3b; top panel). Pancreas of the transgenic male mice showed hyperplasia of the islet cells of Langerhans wherein their number and size were increased (FIG. 3b; top panel). Heart valve showed a mild myxoid change and hypertrophy (FIG. 3b; top panel). Compared to the transgenic male mice, the transgenic female mice showed moderate cell size and volume of peritoneum having less fatty change with only a small amount of vacuolar change (FIG. 3b; second panel). Also, they showed, in a lesser degree, hyperplasia of the islet cells of pancreas and myxoid change of heart valve (FIG. 3b; second panel). The above abnormalities were not observed in the normal control male mice (FIG. 3b; lower panel).

HCCR-1 and ApoE expression profiles were compared with one another by Western blots consisting of several tissues derived from controls and transgenic mice (FIG. 3c). For example, ApoE was highly expressed in the tissues of the control mice such as brain, lung, liver, kidney, intestine, peritoneal cavity, whereas it was minimally expressed or not expressed in the same tissues derived from the transgenic mice. On the other hand, although HCCR-1 level was overexpressed in the transgenic male and female mice, it was minimally expressed or not expressed in the corresponding tissues of the controls. These mutually exclusive expression patterns of HCCR-1 and ApoE remind the data shown in FIGS. 2a and 2b, indicating that they are morphologically regulated in a cooperative manner by signaling pathways which are different but lead opposite biological results.

In the obese male mice, the levels of total cholesterol, HDL cholesterol, LDL cholesterol and triglyceride were greatly increased compared to the normal control mice of the same age (P<0.05). In the obese male mice, the levels of total cholesterol (184.8±5.4 mg/dl), HDL cholesterol (152.9±0.4 mg/dl), LDL cholesterol (46.7±0.7 mg/dl) and triglyceride (21.9±2.3 mg/dl) were increased, respectively, by 4.2 fold, 4.0 fold, 3.8 fold and 2.7 fold in the HCCR-1 transgenic male mice than in the normal mice (FIG. 3d) (P<0.05). In the normal male mice, the levels of total cholesterol, HDL cholesterol, LDL cholesterol and triglyceride were 43.8±1° mg/di, 38.4±1.0 mg/dl, 12.3±0.6 mg/dl and 8.0±0.1 mg/dl, respectively.

However, there was no big difference with respect to ApoE and leptin levels between the obese mice and the normal mice of the same age (FIG. 3d). The ApoE levels in obese male mice, obese female mice and normal control mice were 0.9±0.1 mg/dl, 0.9±0.1 mg/dl and 1.4±0.3 mg/dl, respectively (FIG. 3d). Also, the leptin levels in obese male mice, obese female mice and normal control mice were 0.3±0.1 ng/dl, 0.3±0.1 ng/dl and 0.3±0.1 ng/dl, respectively (FIG. 3d). There was no difference with respect to the serum insulin level between the transgenic obese mice and the control mice (FIG. 3d). The insulin levels in obese male mice, obese female mice and normal control mice were 2.3±0.6 μIU/ml, 2.0±0.3 μIU/ml and 2.0±0.6 μIU/ml, respectively.

From the above, it is apparent that HCCR-1 can be used as a new prognostic marker of breast cancer together with other known prognostic markers. Also, it can be seen that HCCR-1 protein inhibits an antiproliferative action by directly binding to ApoE, which causes tumors, and thus said protein negatively inhibits the ApoE activity. More importantly, it can be seen that obesity occurs in the HCCR-1 transgenic mice, particularly male mice. In connection with the generation of evidences for the relationship between various types of cancer and obesity, the present inventors found that HCCR-1 interacts with ApoE, which is associated with clearance of cholesterol from peripheral cells to liver, and HCCR-1 transgenic mice indicate pathological defects associated with obesity as well as breast cancer. Accordingly, the present invention allows therapeutic strategies to be established for breast cancer and obesity which frequently occur in women, in particular it allows to develop HCCR-ApoE interaction-targeting drugs.

The present invention will be described hereinafter in greater detail by way of non-limiting examples.

### EXAMPLES

### Example 1

### Tissues and Cell Lines

Human normal and cancer tissues were obtained during surgery. Individual consent was obtained from all patients. The use of tissue samples was approved by the ethics committee of the hospital. Mammalian cell lines were obtained from the American Type Culture Collection (ATCC; Manassas, Va.). BT-474, MCF-7 and MDA-MB-231 are the human breast cancer cell lines derived from mammary gland. MCF-7 and MDA-MB-231 cells show a low level of HER2 expression, while BT-474 cells show a high level of HER2 expression. BT-474 and MCF-7 cells are ER-positive and PR-positive. MDA-MB-231 is an ER-negative and PR-negative cell line. The MCF-7 cells have a wild-type p53, while the MDA-MB-231 cells have a null-type p53. The BT-474 cells have a mutant p53.

### Example 2

### Expression Vector Design and DNA Transfection

An expression vector comprising a coding region of HCCR-1 or ApoE was designed as follows.

A SalI segment was first isolated from the prokaryotic expression vector pCEV-LAC comprising HCCR-1 or ApoE cDNA. Then, pcDNA3.1-V5-His (Invitrogen, CA) or pcDNA3.1-Myc-His was treated with BamHI and SalI to create a compatible end having SalI. A Sail segment containing HCCR-1 or ApoE coding sequence was inserted into a XhoI-digested pcDNA3.1.Lipofectamine 2000 (Gibco BRL, Rockville, Md.) which was used to introduce the HCCR-1 or ApoE expression vector into breast cancer cells. Briefly, 2×10⁵ cells were seeded on a 60 mm tissue culture dish (Costar, Cambridge, Mass.). After incubation overnight in a humidified 5% CO₂ incubator, said cells were treated with 150 ml of lipofectamine-DNA complex comprising 15 ml of lipofectamine reagent and 5 mg of DNA. Selection was made for the respective cells which were resistant to 0.6 mg/ml G418. Selected transfectants were screened for HCCR-1 or ApoE expression by Western blot.

### Example 3

### Yeast Two-Hybrid Screening and Beta Galactosidase Analysis

The MATCHMAKER LexA two-hybrid system was used to identify proteins from a human fetal brain MATCHMAKER cDNA library (Clontech, Palo Alto, Calif.) which can bind to a HCCR-1 fusion protein. All experiments were performed in the yeast strain EGY48 (Clontech) transformed with p8op-lacZ, which expresses lacZ and leu genes as reporters. The present inventors inserted a HCCR-1 cDNA segment into a yeast two-hybrid vector (pLexA) (Clontech) containing the LexA DNA-binding domain. Yeast cells expressing the LexA-HCCR-1 were transformed with a human fetal brain cDNA library (Invitrogen) that expresses B42AD fusion proteins. After library transformation, cells were plated on minimal synthetic dropout non-induction medium (Sigma) that selects for both the bait (HCCR-1) and the AD/library plasmids to improve the chances of detecting AD fusion proteins. In order to confirm the interaction between HCCR-1 and binding protein ApoE, plasmids expressing both HCCR-1 and ApoE were co-transformed into yeast cells.

### Example 4

### Co-Transfection and Immunoprecipitation

Cells were transfected with the pcDNA3.1 (Invitrogen, Carlsbad, Calif.) which encodes HCCR-1-V5-His (Invitrogen) fusion protein and ApoE-Myc-His (Invitrogen). After 48 hours, cells were collected and lysed in lysis buffer. The lysates were precleared with preimmune serum (mouse) and protein A-Sepharose at 4° C. for 30 minutes. Protein concentrations were determined using the using the BCA Protein Assay Reagent Kit (PIERCE, Rockford, Ill.) with bovine serum albumin as a standard. Aliquots (1 mg) of precleared cell lysates were incubated with a 1:500 dilution of anti-V5 (Invitrogen) or a 1:250 dilution of anti-Myc (Invitrogen) mAb and 40 ml of a 1:1 slurry of protein A-Sepharose beads (Amersham Biosciences, Uppsala, Sweden) in PBS for 16 hours at 4° C. The immune complexes were collected by centrifugation (2,000×g, 5 min, at 4° C.), washed four times with a buffer (20 mM Tris, pH 7.5, 1 mM EDTA, 1 mM EGTA, 150 mM NaCl, 2 mM Na₃VO₄, 10% glycerol and 1% Nonidet P-40), were subjected to SDS-PAGE, and were Western blotted with a 1:1000 dilution of anti-Myc antibody or a 1:3000 dilution of anti-V5 antibody in TBS.

### Example 5

### Subcellular Localization

Subcellular localization was performed using a mitochondria isolation kit (Pierce, Rockford, Ill.). Briefly, the cells were collected by centrifugation at 850×g for 2 minutes; the pellets were suspended in 800 μl of Reagent A, and then were kept on ice for exactly 2 minutes. 10 μl of Reagent B was added to the suspended solution, and the resulting solution was kept on ice for 5 minutes while vortexing at the maximum speed every minute. 800 μl of reagent C was added to said solution and the tube was inverted several times to mix the solution. The solution was centrifuged at 700×g for 10 minutes at 4° C., and the pellet was used for crude nucleic fraction. The supernatant was centrifuged at 12,000 g for 10 minutes at 4° C. and the supernatant was transferred to a new tube for cytosol fraction. The pellet was washed with 500 μl of reagent C, and used for isolated mitochondria fraction. Each fraction was quantitated with the BCA Protein Assay Reagent Kit (Pierce).

Boiled extracts in sample buffer were subjected to a SDS-PAGE electrophoresis and transferred to nitrocellulose by a standard procedure. Membranes were blocked with 5% skim milk in TBS (pH 7.4) containing 0.05% Tween 20, and incubated with primary antibodies, anti-V5 (Invitrogen, Carlsbad, Calif.), anti-Myc (Santa Cruz Biotechnology, Santa Cruz, Calif.) or anti-VDAC1 (Santa Cruz Biotechnology) for mitochondrial localization marker.

### Example 6

### Immunofluorescence and Fluorescent Microscope

Coverslips were washed in HCl, distilled water (3×) and 100% ethanol (2×). Then, the dried coverslips were coated with 5 μg/ml of Poly-L-Lysine (Sigma-Aldrich Corp., MO) at 37° C. overnight. MCF-7 cells were seeded on the precoated coverslips in a 6-well plate. Then, after one day incubation, the cells were transiently transfected with pEGFP-HCCR-1 and pEGFP-ApoE using LipofectAMINE 2000 (Invitrogen, Carlsbad, Calif.). After incubation for further 24-28 hours, the cells were incubated with 25 nM MitoTracker Orange (Molecular Probes, Eugene, Oreg.) at 37° C. for 15 minutes. The cells were washed in 2 ml of PBS (3×) prior to fixing. Then, the cells on the coverslips were fixed with 4% paraformaldehyde containing 4% sucrose at 4° C. for 10 minutes. The cells were treated with 100% methanol at −20° C. for 1 minute and washed in PBS (3×). For GFP staining, the cells were fixed using ProLong Gold Antifade Reagent (Molecular Probes, Eugene, Oreg.). Fluorescent images were acquired and analysed using a Bio-Rad MRC-1024 MP laser scanning confocal microscope (Bio-Rad, Hercules, Calif.).

### Example 7

### Immunochemical Staining Assay and Staining Interpretation

For Immunochemical staining experiment, paraffin segments (5 μm thick) of human normal breast tissues and breast cancer tissues were used. The segments were incubated with an affinity-purified polyclonal anti-HCCR-1 antibody for 2 hours. Aminoethyl carbozol (AEC) was used as a chromogen. After immunostaining, the segments were stained with hematoxylin. At least 500 tumor cells were counted by a pathologist in the stained regions which were most active for ER, PR and p53. Positive staining for ER, PR and p53 was defined as a nuclear staining. According to generally recognized cutoff values, 10% positive staining of tumor cells was used as a positive result for ER, PR and p53. The HER2 immunochemical staining results were defined according to staining criteria. Positive staining for HER2 was defined as a membrane staining. Protoplasm staining was not considered positive. Tumor cells, which were not immunoreactive (score 0) or showed an incomplete or faint membrane staining (score 1+), were considered negative. When complete weak to moderate membrane staining (weak positive; score 2+) or complete strong membrane staining (strong positive: score 3+) was observed in 10% or more of tumor cells, HER2 was considered positive.

### Example 8

### Northern Blot Analysis

Northern blot analysis was performed to investigate HCCR-1 or ApoE expression in various human tissues using HCCR-1 cDNA or 954-bp full length ApoE cDNA. The mRNA expression level was quantified by comparison with the expression level of beta-actin.

### Example 9

### Detection of ApoE in Culture Supernatant by Sandwich ELISA

In order to study the kinetics of ApoE production during the proliferating phase of breast cancer cells, 10⁶ cells were grown in 75-cm² in DMEM with FBS (10%) for 8 days. Aliquots of 50 ml medium were collected every 24 hours for 8 days and kept at −20° C. until ApoE concentration measurements. ApoE concentrations were determined by sandwich ELISA using a commercial kit (MBL ApoE4/Pan-ApoE ELISA kit, MBL Co., Woburn, Mass.). Human ApoE4 or Pan-ApoE was determined by the MBL ApoE4/Pan-ApoE ELISA kit (MBL Co., Woburn, Mass.). Affinity-purified polyclonal antibodies against ApoE and monoclonal antibodies against ApoE4 were used for the analysis.

### Example 10

### DNA Fragmentation Analysis

HCCR-1- or ApoE-transfected breast cancer cells, pcDNA3.1-only transfected cells and wild type cells were incubated for 3, 5 and 7 days, respectively, and collected. The cells were lysed and digested overnight at 48° C. in lysis buffer containing 100 μg/ml of proteinase K. A 1/5 volume of 5M NaCl and an equal volume of isopropyl alcohol were added to precipitate DNA. The DNA pellet was redissolved in TE buffer and treated with 0.1 mg/ml of RNase A for 1 hour at 37° C. For each sample, 10 μg DNA were fractionated in a 2% agarose gel, stained with ethidium bromide, and visualized under UV ray.

### Example 11

### Production of Transgenic Mice

Transgenic mice were generated using standard pronuclear microinjection as described in Vatten, L. J. & Foss, O. P. Cancer Res 50, 2341-2346 (1990). For microinjection, HCCR-1, the fragment of a transgene was separated free from the vector backbone of pcDNA3. 1-V5-His by Nru I and Xmn I double digestion. The injected fragments of CMV-HCCR-1-bGH were isolated and purified using electroelution and dialysis, diluted to a final concentration of 2 ng/ml DNA injection buffer (10 mM Tris/0.1 mM EDTA, pH 7.4), and microinjected into the pronuclei of one cell-stage fertilized embryos derived from C57BL/6N (Charles River Japan). Then 20-25 injected DNA fertilized eggs that survived microinjection were implanted into the oviducts of one recipient CD-1 (Charles River Japan) mouse as described 2-3 hours after injection or on the next day. Potential transgenic founder animals were weaned at 3 week of age, and identified by screening mouse tail genomic DNA prepared with standard protocols for the presence of HCCR-1 transgene using PCR, and allowed to grow in a wild type manner to develop an persistent line.

### Example 12

### Statistical Analysis

Body weight and serum concentrations of ApoE, total cholesterol, HDL, LDL, triglycerides, leptin and insulin were expressed as mean±SD. The t-test and Dunnett's multiple comparison were used for all statistical analysis.

As described hereinbefore, HCCR-1 of the present invention has superior effect as a prognostic marker for breast cancer and/or a composition for inducing obesity.

1. A composition for use as a prognostic marker for breast cancer, comprising FICCR-1 protein.

2. The composition according to claim 1, wherein said composition further comprises one or more agents selected from the group consisting of ER, PR, p53 genotype and HER2 protein.

3. The composition according to claim 1, wherein the sequence of said HCCR-1 protein is asset forth in SEQ ID NO:1.

4. A composition for inducing obesity, comprising HCCR-1 protein.

5. A transgenic obese non-human mammal wherein said mammal has been transformed with HCCR-1 protein.

6. The transgenic, obese non-human mammal according to claim 5, wherein said mammal is selected from the group consisting of mouse, rat, rabbit, sheep, bovine, goat and porcine.

7. The composition or mammal according to claim 4, wherein the sequence of said HCCR-1 protein is as set forth in SEQ ID NO:1.

8. The composition or mammal according to claim 5, wherein the sequence of said HCCR-1 protein is as set forth in SEQ ID NO:1.
